(19) United States
(12) Patent Application Publication
(10) Pub. No.: US 2015/0182527 A1
(43) Pub. Date: Jul. 2, 2015

(54) COMPOSITIONS, DEVICES AND METHODS OF TREATING INFECTIONS

(71) Applicant: COOK MEDICAL TECHNOLOGIES LLC, Bloomington, IN (US)

(72) Inventors: Steven L. Pendleton, Spencer, IN (US); Will C. McRoy, JR., West Lafayette, IN (US)

(73) Assignee: COOK MEDICAL TECHNOLOGIES LLC, Bloomington, IN (US)

(21) Appl. No.: 14/572,057

(22) Filed: Dec. 16, 2014

Related U.S. Application Data
(60) Provisional application No. 61/923,003, filed on Jan. 2, 2014.

(51) Int. Cl. A61K 31/506; A61L 29/14; A61L 29/16; A61K 31/14; A61K 31/4196

(52) U.S. Cl. CPC A61K 31/506; A61K 31/14; A61K 31/4196; A61L 29/16; A61L 29/146; A61L 2300/404

(57) ABSTRACT

One aspect provides a composition including a quaternary ammonium salt and an azole. The azole and the quaternary ammonium salt can be present in proportions providing a synergistic effect against a fungal organism. In one embodiment, the composition also includes a pharmaceutically acceptable carrier. Another aspect provides implantable medical devices containing a composition including a quaternary ammonium salt and an azole. In certain embodiments, the combination is present in an elutable form. Another aspect provides methods of treating a human or veterinary patient for an infection including administrating a composition including a quaternary ammonium salt and an azole.

### RELATED APPLICATIONS

The present application claims priority from U.S. Provisional Patent Application Ser. No. 61/923,003 filed Jan. 2, 2014, the contents of which are hereby incorporated by reference in their entirety.

### TECHNICAL FIELD

The present invention relates generally to compositions, devices and methods for treating infections. One embodiment provides a composition including a quaternary ammonium salt and an azole. Another embodiment provides an implantable medical including a quaternary ammonium salt and an azole. Yet another embodiment provides a method of preventing or treating an infection in a patient including administrating a composition including a quaternary ammonium salt and an azole.

### BACKGROUND

Infection of patients by microbial organisms, including infection associated with medical implants, represent a major health care problem. A significant number of patients admitted to hospitals develop a hospital acquired infection. Such infections are a leading cause of death. Four common types of infections are urinary tract infection, surgical site infection, respiratory tract infection and bloodstream infection. A significant percentage of these infections are related to microbial colonization of implanted medical devices such as Foley catheters, endotracheal and tracheostomy tubes, and vascular infusion catheters. Although any infectious agent can infect medical implants, bacteria, such as Staphylococci, Enterococci, Gram Negative Aerobic Bacilli and Pseudomonas account for a significant portion of such infections.

Infections can also be caused by fungal agents. Fungal infections occur in many patients each year and evidence suggests the rate of such infections is increasing. Fungi can infect almost any part of the body including the skin, nails, respiratory tract, urogenital tract or alimentary tract. Fungal infections can also be systemic. Elderly patients and those with a weakened immunity have a higher risk of fungal infections.

Although several species of fungi are potentially pathogenic in humans, Candida, especially Candida albicans and Candida glabrata, is responsible for most fungal infections. Candida, which is normally present within the human body, is usually harmless. However, it can cause symptoms when a weakened immune system or other factors allow it to grow unabated. The increased use of antibiotics is a major factor contributing to an increase in the resistance of fungal infections to conventional antimicrobial agents. Such fungal infections can range in severity from superficial to life-threatening.

In an attempt to combat device colonization resulting in infection of the patient, implantable devices are sometimes coated with antimicrobial agents. Catheter lock solutions including antimicrobial agents can also be used to combat such colonization. However, such devices can become colonized by microbes resistant to the antimicrobial in the coating or lock solution. Such antimicrobial-resistant microbes can make infection control more complex. There is the need for improved antimicrobial agents, including combinations of such agents that provide improved treatment of such infections.

### SUMMARY

In one aspect, the present invention provides a composition including a quaternary ammonium salt and an azole. In one embodiment, the azole and the quaternary ammonium salt are present in proportions providing a synergistic effect against a fungal organism. The composition can also include a pharmaceutically acceptable carrier. In various embodiments, the azole is fluconazole and the quaternary ammonium salt is benzethonium chloride or benzalkonium chloride. The fungal organism can be a species of the genus Candida, for example, Candida albicans.

Another aspect of the invention provides a method of preventing or treating an infection in a patient including administrating a therapeutically effective amount of a composition including a quaternary ammonium salt and an azole to the patient. In one embodiment, the azole and the quaternary ammonium salt are present in proportions providing a synergistic effect against a fungal infection. The fungal infection can be, for example, a urinary tract infection, a surgical site infection, a respiratory tract infection or bloodstream infection. The fungal organism causing the infection can be, for example, a species of the genus Candida, for example, Candida albicans. The composition can be administered systemically, such as by intravenous administration. Alternatively, the composition can be administrated by non-vascular infusion, such as infusion to the urinary system, for example, the bladder. The composition can also be administered as, for example, an elutable component of an implantable medical device.

Another aspect of the invention provides an implantable medical device including a body structure containing a composition including a quaternary ammonium salt and an azole. In one embodiment, the azole and the quaternary ammonium salt are present in proportions providing a synergistic effect against a fungal organism. In another embodiment, the composition is contained within a polymeric material in a manner that allows for controlled elution of the composition upon implantation. The azole can be a triazole, for example, fluconazole or voriconazole. The quaternary ammonium salt can be, for example, benzethonium chloride or benzalkonium chloride.

In other aspect of the invention provides a method for manufacturing a medical device. In one embodiment, the method includes forming a mixture comprising a polymeric material, a quaternary ammonium salt and an azole, heating the mixture to a temperature sufficient to melt the polymeric material and extruding the mixture to form at least a portion of the body of the medical device. The polymeric material can include polyurethane.

### BRIEF DESCRIPTION OF THE DRAWINGS

FIG. 1 depicts a partial cross-sectioned end view of an illustrative embodiment of an implantable medical device.

FIG. 2 depicts a partial cross-sectioned end view of another embodiment of the implantable medical device.

FIG. 3 depicts a partial cross-sectioned end view of yet another embodiment of the implantable medical device.

FIG. 4 depicts a plan view of a Foley catheter.

FIG. 5 depicts a plan view of a ureteral stent.

FIG. 6 is an illustration showing the efficiency of combinations of fluconazole (Fluc) and benzethonium chloride (Bt-Cl) against C. albicans (Panel A) and C. glabrata (Panel B) at 42 hours post inoculation in TSB media.

FIG. 7 is an illustration showing the efficiency of combinations of fluconazole (Fluc) and benzethonium chloride (Bt-Cl) against C. albicans (Panel A) and C. glabrata (Panel B) at 48 hours post inoculation in RPMI media.

FIG. 8 is an illustration showing the efficiency of combinations of fluconazole (Fluc) and benzalkonium chloride (Balk-Cl) against C. albicans (Panel A) and C. glabrata (Panel B) at 48 hours post inoculation in TSB media.

FIG. 9 is an illustration showing the efficiency of combinations of voriconazole (Vor) and benzethonium chloride (Bt-Cl) against C. albicans (Panel A) and C. glabrata (Panel B) at 48 hours post inoculation in TSB media.

FIG. 10 is an illustration summarizing the interactions between the compounds tested.

### DETAILED DESCRIPTION OF THE PREFERRED EMBODIMENTS

### Definitions

Unless otherwise defined, all technical and scientific terms used herein have the same meaning as commonly understood by one of ordinary skill in the art to which this invention pertains. In case of conflict, the present document, including definitions, will control. Preferred methods and materials are described below, although methods and materials similar or equivalent to those described herein can be used in the practice or testing of the present invention. All publications, patent applications, patents and other references mentioned herein are incorporated by reference in their entirety.

The uses of the terms “a” and “an” and “the” and similar references in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”, “for example”) provided herein, is intended merely to better illuminate the invention and does not pose a limitation on the scope of the invention unless otherwise claimed. No language in the specification should be construed as indicating any non-claimed element as essential to the practice of the invention.

As used herein the terms “comprise(s),” “include(s),” “having,” “has,” “can,” “contain(s),” and variants thereof, are intended to be open-ended transitional phrases, terms, or words that do not preclude the possibility of additional acts or structures. The present invention also contemplates other embodiments “comprising,” “consisting of” and “consisting essentially of,” the embodiments or elements presented herein, whether explicitly set forth or not.

As used herein, the term “implantable” refers to an ability of a medical device to be positioned at a location within a body, such as within a body vessel. Furthermore, the terms “implantation” and “implanted” refer to the positioning of a medical device at a location within a body, such as within a body vessel.

The term “bioabsorbable” is used herein to refer to materials that dissipate upon implantation within a body, independent of which mechanisms by which dissipation can occur, such as dissolution, degradation, absorption and excretion. A “non-bioabsorbable” or “biostable” material refers to a material, such as a polymer or copolymer, which remains in the body without substantial bioabsorption.

The term “adapted” for introduction into a human or veterinary patient is used herein to refer to a device having a structure that is shaped and sized for introduction into a human or veterinary patient.

As used herein, the term “body vessel” means any body passage or lumen, including but not limited to blood coronary or peripheral vessels, esophageal, intestinal, biliary, urethral and ureteral passages.

As used herein, the term “bioactive” refers to any agent that produces an intended therapeutic effect on the body to treat or prevent conditions or diseases.

A “therapeutically-effective amount” as used herein is the minimal amount of a bioactive or a combination of bioactives (e.g. a combination of a quaternary ammonium salt and an azole) which is necessary to impart therapeutic benefit to a human or veterinary patient.

Compositions for Treating Infections and Implantable Medical Devices including such Compositions

For the purpose of promoting an understanding of the principles of the invention, reference will now be made to embodiments, some of which are illustrated in the drawings, and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

In the discussions that follow, a number of potential features or selections of the quaternary ammonium salt, azole, amounts and ratios of composition components, implantable device structure, or other aspects, are disclosed. It is to be understood that each such disclosed feature or features can be combined with the generalized features discussed, to form a disclosed embodiment of the present invention.

Certain aspects of the present invention relate to compositions including a quaternary ammonium salt and an azole. Other aspects relate to an implantable medical devices including such a combination or to methods of treating a human or veterinary patient by administrating such a combination. The composition can also include a pharmaceutically acceptable carrier. In certain embodiments, the quaternary ammonium salt and the azole are present in proportions that provide a synergistic effect against a fungal organism.

In various embodiments, the quaternary ammonium salt is benzethonium chloride, benzalkonium chloride, methylbenzethonium chloride, cetalkonium chloride, cetylpyridinium chloride, cetrimonium, cetrimide, dofanium chloride, tetraethylammonium bromide, didecyldimethylammonium chloride or domiphen bromide. In some embodiments, the azole is an imidazole, such as bifonazole, butoconazole, clotrimazole, econazole, fenticonazole, isoconazole, ketoconazole, miconazole, omoconazole, oxiconazole, sertaconazole, sulconazole or tioconazole. In other embodiments, the azole is a triazole, such as albaconazole, fluconazole, isavuconazole, itraconazol, posaconazole, ravuconazole, terconazole or voriconazole. In yet other embodiments, the azole is a thiazole, such as abafungin. In certain embodiments, the quaternary ammonium salt is benzethonium chloride and the azole is fluconazole. Compositions including combinations of two or more of the quaternary ammonium salts with two or more azoles are also within the scope of the present embodiments.

In certain embodiments, the quaternary ammonium salt and azole are present at a ratio of between 1:300 w/w and 1:1 w/w, 1:200 w/w and 1:1 w/w, 1:100 w/w and 1:1 w/w, 1:50 w/w and 1:1 w/w, 1:20 w/w and 1:1 w/w or 1:10w/w and 1:1 w/w. In other embodiments, the quaternary ammonium salt and azole are present at a ratio of between 300:1 w/w and 1:1 w/w, 200:1 w/w and 1:1 w/w, 100:1 w/w and 1:1 w/w, 50:1 w/w and 1:1 w/w, 20:1 w/w and 1:1 w/w or 10:1w/w and 1:1 w/w. In yet other embodiments, the quaternary ammonium salt and azole are present at a ratio of between 300:1 w/w and 1:300 w/w, 200:1 w/w and 1:200 w/w, 100:1 w/w and 1:100 w/w, 50:1 w/w and 1:50 w/w, 20:1 w/w and 1:20 w/w or 10:1 w/w and 1:10 w/w.

In one embodiment the synergistic effect of the quaternary ammonium salt and azole is determined by a microdilution method adapted from the method outlined in CLSI, M27-A3 (2008), —Reference Method for Broth Dilution Antifungal Susceptibility Testing of Yeasts; Approved Standard—3rd Edition.

In certain embodiments, synergistic effect of the quaternary ammonium salt and azole is such that the amount of one or both of the compounds used to treat an infection can be reduced at least 5%, 10%, 15%, 20%, 25%, 30%, 40%, 50%, 70%, 90%, 95% or 98% compared to the amount required in the absence of the synergistic effect.

The medical device including the composition can be any of a wide variety of devices having an implantable structure adapted for temporary or permanent implantation in a human or veterinary patient. Medical devices having structures implantable in a body passage will often be used. The body passage can be, for example, a passage of the alimentary system, the urogenital system, the biliary system, or the cardiovascular system. The medical device can also be a device that is adapted for at least partial implantation at a surgical site.

Medical devices including a structure implantable in the urogenital system are preferred, including, for example, those implantable in the ureter, bladder or urethra. In other embodiments, the device has a structure implantable in a vessel of the cardiovascular system. The vessel can be a tubular passage such as an artery or vein, or can be a larger chamber such as a ventricle or atrium of the heart. Implantable medical devices that include structures that span or bridge between bodily passages are also contemplated. The device can be adapted to be entirely or only partially implanted in a bodily passage.

By way of example, the medical device can be or include a catheter, a balloon catheter, a wire guide, a stent, a stent graft, a coil, a needle, a graft, a filter, a balloon, a cutting balloon, a scoring balloon, or any combination of these. The catheter can be, for example, a urethral catheter, a catheter for nephrostomy drainage, a catheter for nasal pancreatic drainage, a catheter for suprapubic drainage, or a nasal biliary drainage catheter. The stent can be, for example, a coronary or peripheral vascular stent, a urethral stent, a prostatic stent, a biliary stent, a pancreatic stent. These and other variations in the implantable medical device and its associated procedure for introduction will be apparent to those skilled in the pertinent art from the descriptions herein.

The implantable medical device can be made from any suitable material or combination of materials. Illustratively, the implantable medical device can include a metal such as stainless steel, tantalum, titanium, nitinol, cobalt, chromium, nickel, molybdenum, manganese, gold, platinum, inconel, iridium, silver, tungsten, elgiloy, alloys or mixtures of two or more of any of these, or another biocompatible metal; carbon or carbon fiber; a calcium-containing inorganic material such as a ceramic; a material composed of ceramic and metallic components (cermet); or a polymeric material. The material of construction for the implantable medical device structure can be biodegradable or biostable.

The implantable medical device can include biostable polymers, for instance cellulose acetate, cellulose nitrate, silicone, polyethylene terephthalate, polyurethane, polyamide, polyester (e.g. Nylon), polyorthoester, polyanhydride, polyether sulfone, polycarbonate, polypropylene, high molecular weight polyethylene, and polytetrafluoroethylene, or mixtures of these. Bioabsorbable polymers can also be used, including for instance polylactic acid (PLA), polyglycolic acid (PGA), poly(lactic-co-glycolic acid) (PLGA), polyanhydride, polycaprolactone, polyhydroxybutyrate valerate, hydrogels, natural polymers such as fibrin, collagens, extracellular matrix (ECM) materials, dextrans, polysaccharides and hyaluronic acid, or co-polymers and/or mixtures of two or more of these.

The implantable device can include one or more bioabsorbable metals. For example, bioabsorbable metal devices, such as stents, can incorporate bioabsorbable materials such as magnesium, titanium, zirconium, niobium, tantalum, zinc, silicon, lithium, sodium, potassium, calcium, iron, manganese, yttrium, rare earth metals, such as neodymium, or alloys and/or mixtures of two or more of these materials. Some preferred metallic bioabsorbable material alloy compositions include lithium-magnesium, sodium-magnesium, and zinc-titanium. Further details of bioabsorbable metals useful in the manufacture of implantable devices are described in U.S. Patent Publication Number 2010/0262221, the contents of which are incorporated by reference.

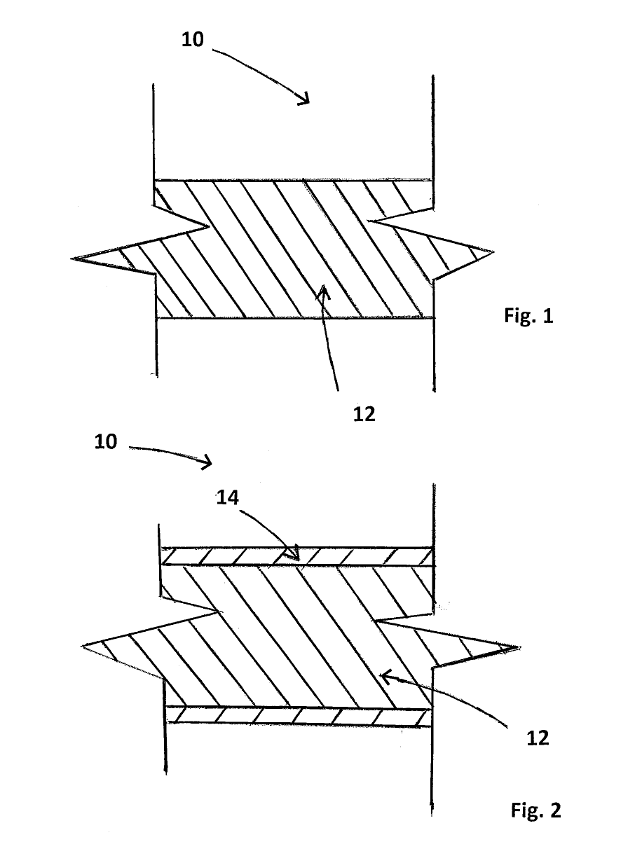

With reference now to FIG. 1, this figure shows an implantable medical device 10 including a structure 12 adapted for introduction into a human or veterinary patient. For clarity, only a portion of the structure is shown. By way of example, device 10 can be configured as a catheter particularly adapted for insertion into the urinary system of the patient, such as into the urethra.

In certain embodiments, structure 12 includes a polymer incorporating a composition including a quaternary ammonium salt and an azole. Such devices can be manufactured, for example, by impregnating structure 12 with a mixture of a quaternary ammonium salt and an azole. Methods of impregnating polymeric structures are described in U.S. Patent Publication Number 2006/0025726, the contents of which are incorporated by reference. A mixture of a quaternary ammonium salt and an azole can be impregnated into the polymer structure or each of the two components can be added separately. In some embodiments, one or more of the quaternary ammonium salt and the azole can be impregnated into separate regions of structure 12.

In other embodiments, the quaternary ammonium salt and the azole are incorporated into structure 12 during the formation of the structure, for example, during a molding process. Typically, a mixture of the quaternary ammonium salt and the azole is mixed with a polymeric material, which is then cross linked or dipped cast to form the required structure. The device can also be formed by incorporating one of the components into the structure before polymerization and then impregnating the remaining component, as discussed above, after the structure is formed.

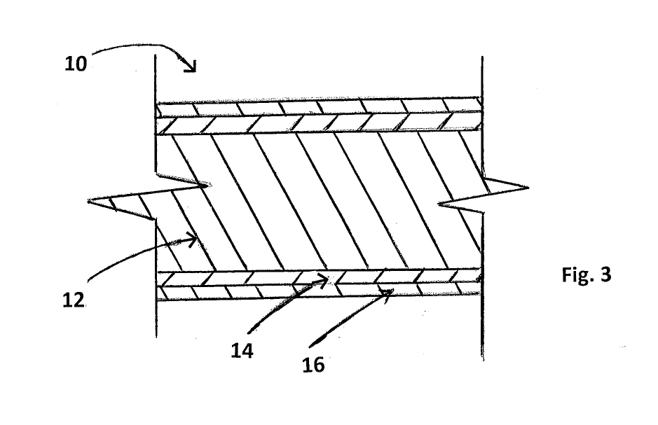

FIG. 2 illustrates another embodiment showing an implantable medical device 10 including a structure 12 and a coating 14 covering at least part of the structure. A mixture of the quaternary ammonium salt and the azole can be present in coating 14. In certain embodiments, a carrier material, such as a biostable or bioabsorbable polymer, is also present in coating 14. The carrier material can act to control the release of the composition when the device is implanted. For example, the carrier material can control the release of the composition such that it is eluted from the device over a longer time period than would be the case if the carrier material was not present. Alternatively, the carrier material can act such that the mixture is eluted more quickly than it would if the carrier material was not present. In other embodiments, no carrier material is present. For example, the coating can include the quaternary ammonium salt and azole in the absence of any polymer or other material that controls the elution of the composition from the device. In certain embodiments, the coating includes only the quaternary ammonium salt and the azole. In other embodiments, the layer containing the quaternary ammonium salt and azole is the outermost layer of the coated device.

With reference now to FIG. 3, this figure shows an implantable medical device 10 including a structure 12, a coating 14 covering at least part of the structure, and an overcoat layer 16 covering at least a portion of coating 14. Layer 16 can be formed from a porous material, for example a polymer, or from a bioabsorbable material, such as a bioabsorbable polymer. Layer 16 can also act to control the release of the composition from the device and can also provide protection for the underlying layer(s) during implantation of the device.

The implantable device can include additional coating layers. For example, the device can include multiple coating layers, each containing the composition. In other embodiments, other bioactive materials can be present, either in the same layer as the mixture or in separate layers. The layers can be separated from each other by layers containing no elutable component, for example by porous polymer layers. Alternatively, multiple layers containing one of both of the quaternary ammonium salt and the azole and/or other bioactives can be deposited directly on top of each other. In some embodiments, the quaternary ammonium salt and the azole are contained in separate layers. In other embodiments, one of the quaternary ammonium salt and the azole can be contained within structure 12 while the other component can be included in one or more of the coating layers. Both of the quaternary ammonium salt and the azole can be included within the structure of the device and in at least one coating layer. In yet other embodiments, structure 12 can include apertures, such as holes or wells, containing at least one of the quaternary ammonium salt and the azole. Further examples of implantable structures incorporating coatings including elutable bioactives that are applicable to the present embodiments are disclosed in U.S. Pat. No. 7,410,665, the contents of which are incorporated by reference.

Coatings 14 and 16 can be applied to the medical device in any known manner. For example, a coating can be applied by spraying, dipping, pouring, pumping, brushing, wiping, vacuum deposition, vapor deposition, plasma deposition, electrostatic deposition, ultrasonic deposition, epitaxial growth, electrochemical deposition or any other method known to those skilled in the art.

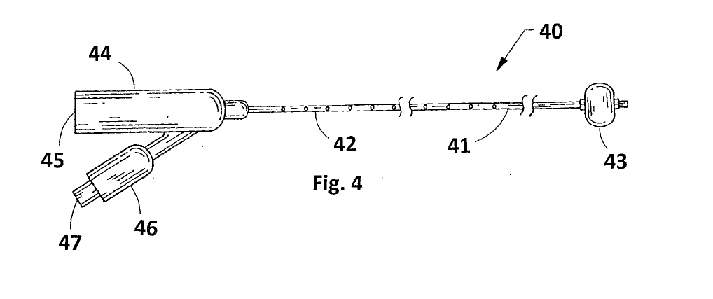

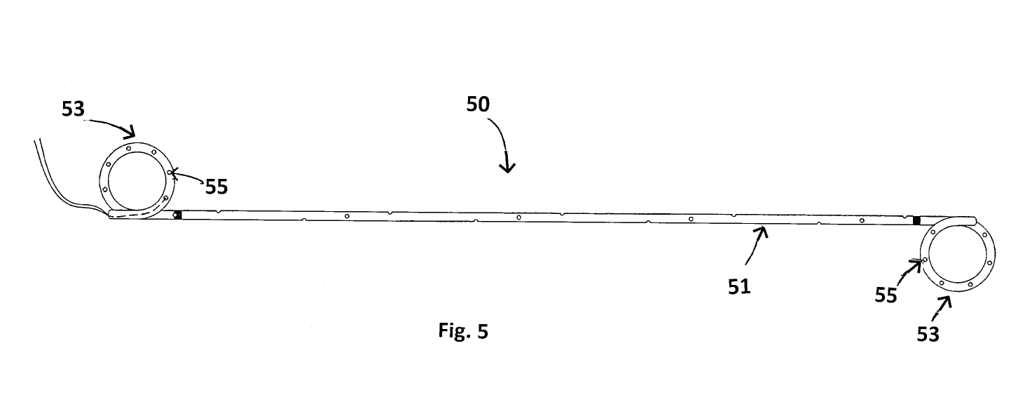

FIGS. 4 and 5 illustrate examples of medical devices applicable for use with the present invention. FIG. 4 depicts a Foley catheter 40, which includes elongated element 41 for draining urine from a urinary bladder of a patient. The Foley catheter has a constant cross section or diameter for most of the length of elongated element 41, except for a retention balloon 43. Balloon 43 is placed into the patient's bladder and is then inflated using fitting 46 and inflation lumen 47. Urine is drained from the patient through outlet 45 and outlet fitting 44, which can be used to connect to a container, such as a drainage bag. Elongated element 41 can be at least partly formed of a polymer or elastomer.

FIG. 5 depicts a view of a ureteral stent. Ureteral stent 50 includes an elongated element 51 connecting curled ends 53. The ureteral stent is implanted such that one of the curled ends 53 is placed in at the top end of the ureter, near the kidney. The second curled end is positioned in the bladder. Urine enters the stent through holes 55 at the curled end positioned near the kidney and travels through a lumen within elongated element 51 to exit the stent through holes 55 at the second curled end and into the bladder. Such Foley catheters or ureteral stents can contain a region that is impregnated, coated, or otherwise includes the quaternary ammonium salt and the azole.

Another aspect of the invention provides a pharmaceutical composition including the quaternary ammonium salt and the azole and a pharmaceutically-acceptable carrier and the administration of such a pharmaceutical composition to a subject in a manner commensurate with treatment for symptoms associated with an infection. Such pharmaceutical compositions can be formulated, for example, by employing conventional solid or liquid vehicles or diluents, as well as pharmaceutical additives of a type appropriate to the mode of desired administration (for example, excipients, binders, preservatives, stabilizers, flavors) according to techniques such as those well known in the art of pharmaceutical formulation.

The pharmaceutical compositions can be administered by any suitable means. For example, they can be administered by subcutaneous, intravenous, intramuscular, or intracisternal injection or infusion techniques (for example, as sterile injectable aqueous or non-aqueous solutions or suspensions; as sterile injectable aqueous or non-aqueous solutions delivered by an infusion catheter); nasally, such as by inhalation spray; or topically, such as in the form of a cream or ointment; orally and in dosage unit formulations containing non-toxic, pharmaceutically-acceptable vehicles or diluents. Such pharmaceutical compositions can, for example, be administered in a form suitable for immediate release or extended release. Immediate release or extended release can be achieved by the use of suitable pharmaceutical compositions, or, particularly in the case of extended release, by the use of devices such as subcutaneous implants or osmotic pumps. The pharmaceutical compositions can also be administered in the form of liposomes.

Oral pharmaceutical compositions generally include an inert diluent or an edible carrier. They can be enclosed in gelatin capsules or compressed into tablets. For the purpose of oral therapeutic administration, the pharmaceutical composition can include excipients and be in the form of tablets, troches, or capsules. Oral compositions can also be prepared using a fluid carrier for use as a mouthwash, wherein the compound in the fluid carrier is applied orally. Pharmaceutically compatible binding agents, and/or adjuvant materials can be included. Tablets, pills, capsules, troches and the like can contain, for example, the following ingredients, or compounds of a similar nature: a binder such as microcrystalline cellulose, gum tragacanth or gelatin; an excipient such as starch or lactose, a disintegrating agent such as alginic acid or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

### Methods of Preventing or Treating a Microbial Infection

Another aspect of the present invention provides methods of delivering a therapeutically effective amount of the quaternary ammonium salt and the azole to the tissue of a patient for the purposes to treating or preventing an infection. The patient can be a human or veterinary patient. In one embodiment, the patient is a mammalian patent, for example a human patient.

The quaternary ammonium salt and the azole can be delivered as a pharmaceutical composition, such as those disclosed above, or in combination with the implantable devices disclosed above. The pharmaceutical composition can be delivered directly to a vessel of a patient. In one embodiment, the pharmaceutical composition is delivered to the urinary tract of a patient by, for example, infusion through a catheter.

In certain other embodiments, the method includes the steps of inserting an implantable medical device having any of the configurations described above into the patient and maintaining the device in position for a time sufficient to deliver the composition to the tissue of the patient. For example, the composition can be delivered locally to a passage of the alimentary system, the urogenital system, the biliary system, or the cardiovascular system by elution from the device. In other embodiments, the composition can be delivered as a component of a catheter lock solution.

In another embodiment, the device is a urethral or ureteral catheter or stent, for example, a Foley catheter introduced into the patient's bladder via the urethra for the drainage of urine. In such an embodiment, the quaternary ammonium salt and the azole, can be included in or on the device to prevent or treat any infection associated with the implantation of the device. In yet another embodiment, the composition is delivered, for example, in a catheter lock/flush solution.

The following examples illustrate the present invention. The examples and embodiments described herein are for illustrative purposes only and modifications or changes in light thereof will be suggested to one skilled in the art without departing from the scope of the present invention.

### EXAMPLES

### Example 1

### The Efficacy of Azole and Quaternary Ammonium Salt Combinations Against Select Yeast

Interactions between the selected azoles and quaternary ammonium salts (quats) were characterized via changes in minimum inhibitory concentration (MIC) using a checkerboard method in 96-well plates. This method is adapted from the microdilution method outlined in CLSI, M27-A3 (2008), —Reference Method for Broth Dilution Antifungal Susceptibility Testing of Yeasts; Approved Standard—3^{rd} Edition.

Briefly, X and Y axes were established on the plates to serve as MIC controls for the compounds acting individually. Two-compound interactions were evaluated at the X-Y axis intersection of the two single-drug concentration axes. A dilution series (typically a two-fold dilution series) of each compound across the selected range was made at 4× the final concentration in culture media.

Frozen concentrated stock compound solutions were used as the starting point in making the dilution series. A voriconazole stock solution was dissolved in DMSO while benzethonium chloride, benzalkonium chloride and fluconazole were dissolved in sterile water. One-quarter of the final well volume of the 4× stock was added to the appropriate wells. For wells with a second compound, one-quarter of the final well volume of the second compound was also added. An equivalent volume of culture media was added to wells in which only a single compound was tested.

Final well volume was either 100 μl or 200 μl depending on assay run. After addition of the 4× stocks and/or media, one-half of the final well volume of 5×10³ CFU/mL yeast inoculum was added to each test well. Yeast inoculums were made from a 0.5 McFarland-matched culture that had been growing for 4-5 hours at 37° C. in trypticase soy broth (TSB). Each assay plate also included inoculum-only growth control wells (half the final well volume of inoculum plus the other half culture media) and culture media-only control wells. The inoculated plates were cultured aerobically in a humidified 36° C. room air incubator for 18 hours or 24 hours (±1 hour), depending on assay run, at which time growth was scored. The scored plates were then returned to the incubator for an additional 24 hours (±1 hour) of culturing and scored a second time (at 42 or 48 hours total culturing time).

Unless stated otherwise, yeast growth was scored using the subjective five tier numerical score (NS) scale established in CLSI M27-A3: 4=no reduction in growth relative to control, 3=slight reduction in growth (-25% reduction), 2=prominent decrease in growth (˜50% reduction), 1=slightly hazy growth (-75% reduction), 0 =no visible growth. Growth is scored relative to the on-plate inoculum control.

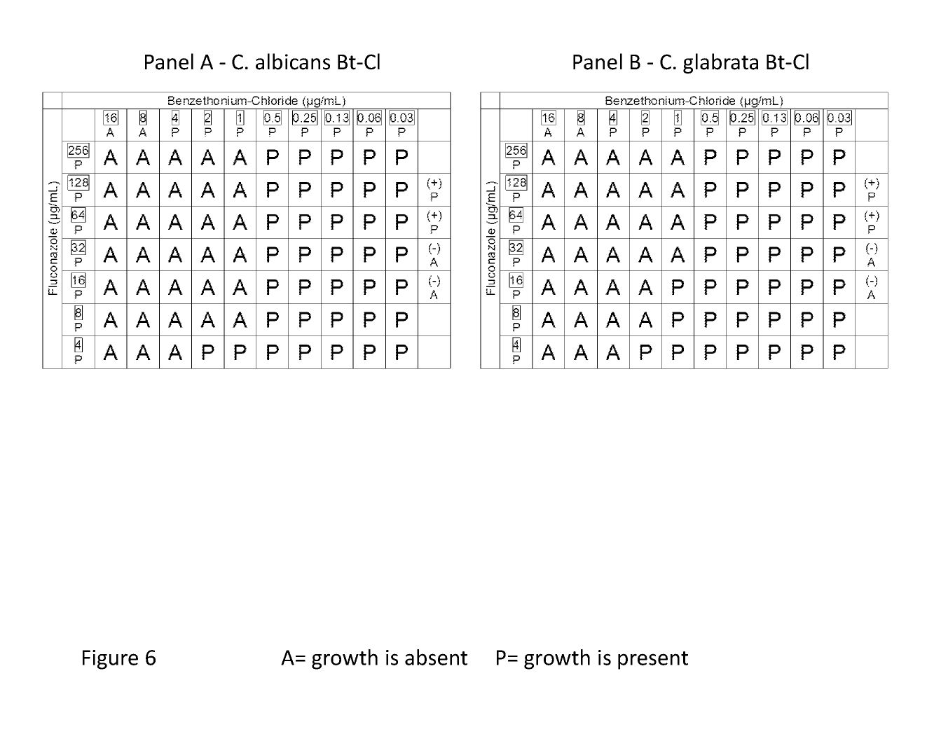

FIG. 6 shows the efficiency of combinations of fluconazole and benzethonium chloride against C. albicans (Panel A) and C. glabrata (Panel B) at 42 hours post inoculation in TSB media. The final concentration of benzethonium chloride ranged from 16 micrograms/ml to 0.031 micrograms/ml and that of fluconazole from 256 micrograms/ml to 4 micrograms/ml. In this assay, growth was scored as present or absent based on visual observation and not using the NS system.

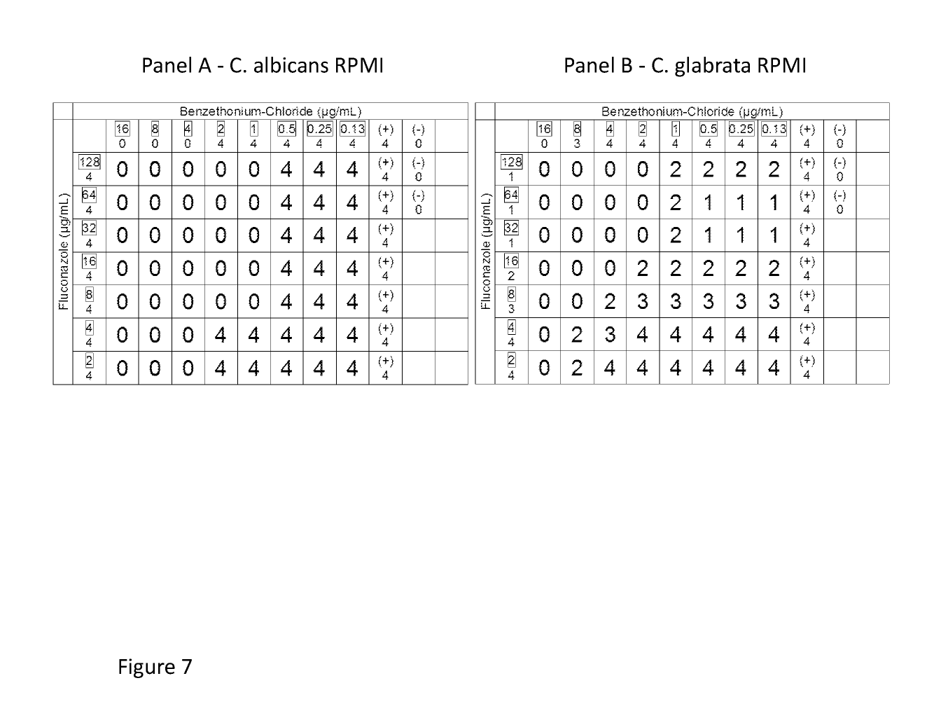

FIG. 7 shows the efficiency of combinations of fluconazole and benzethonium chloride against C. albicans (Panel A) and C. glabrata (Panel B) at 48 hours post inoculation in RPMI media. Growth was scored using the NS system.

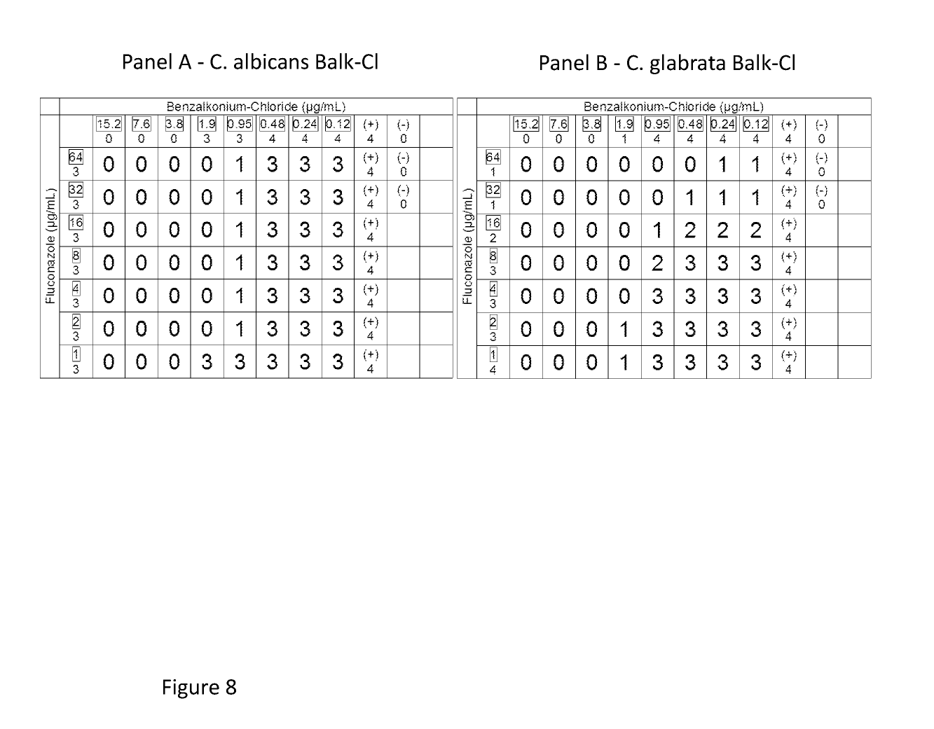

FIG. 8 shows the efficiency of combinations of fluconazole and benzalkonium chloride against C. albicans (Panel A) and C. glabrata (Panel B) at 48 hours post inoculation in TSB media. Growth was scored using the NS system.

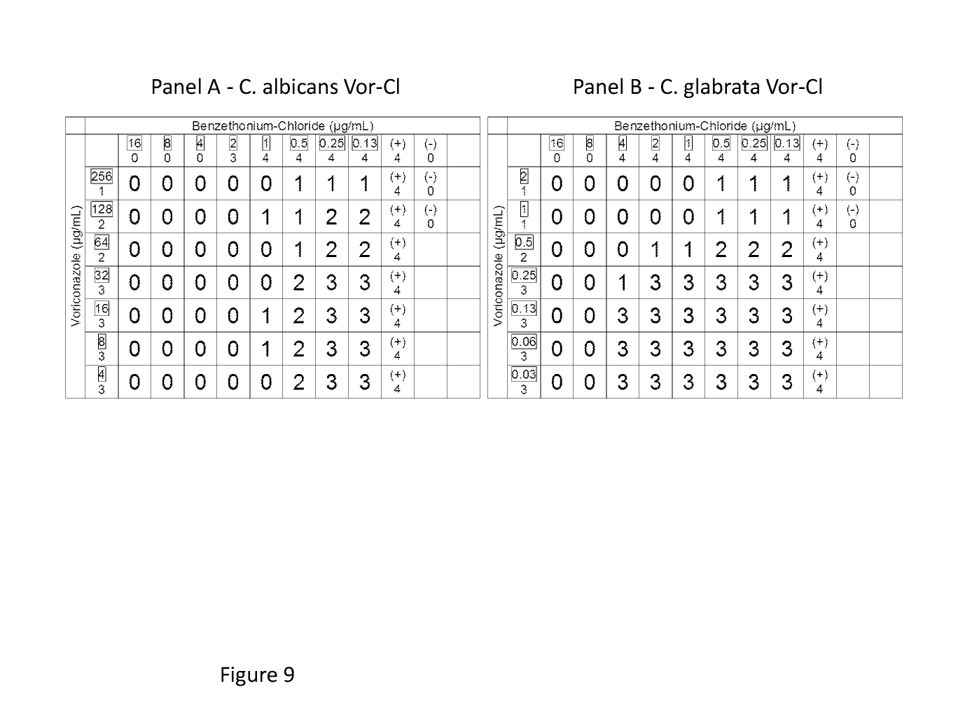

FIG. 9 shows the efficiency of combinations of voriconazole and benzethonium chloride against C. albicans (Panel A) and C. glabrata (Panel B) at 48 hours post inoculation in TSB media. Growth was scored using the NS system.

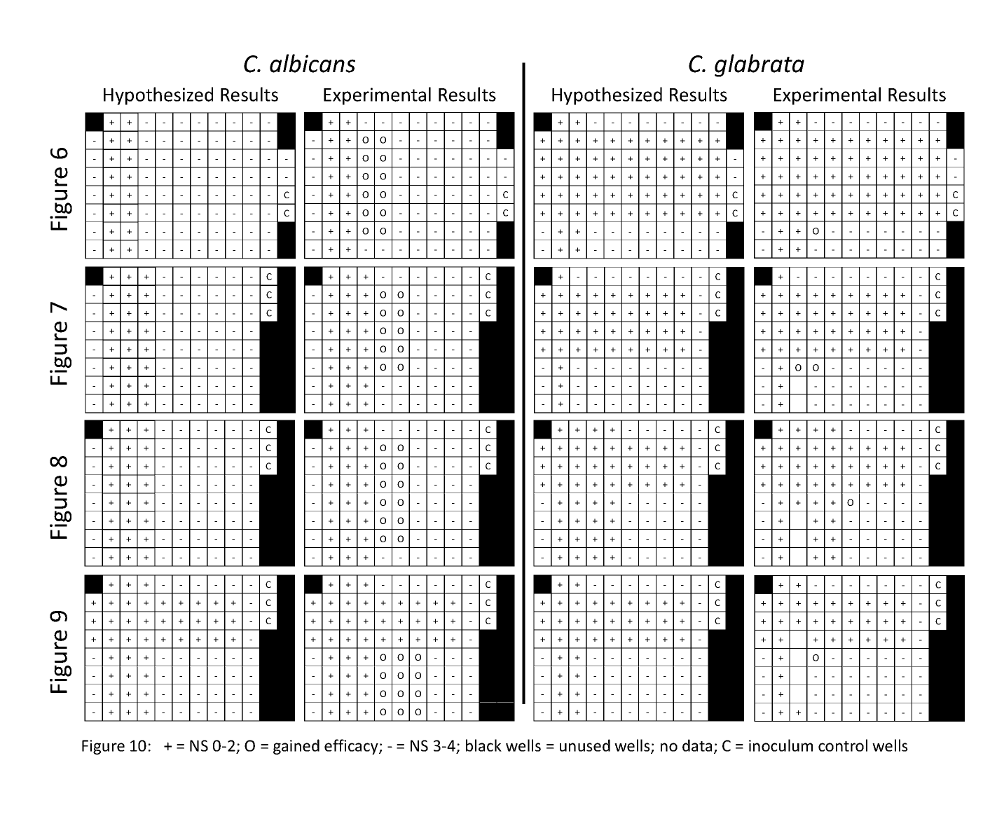

FIG. 10 summarizes the interactions between compounds. For each yeast species, the left-hand column presents the hypothesized results if the compounds were not interacting (i.e. only the individual compound MICs define the growth breakpoints). The right-hand column presents the experimental results as either efficacious (“+”; i.e. no growth) or non-efficacious (“−”; i.e. growth). To simplify the NS scoring used in FIGS. 7-9, the scores were grouped as follows: NS 0-2=+, NS 3-4=−. The “o” boxes highlight the gained efficacy when combining the compounds (i.e. wells that would be hypothesized to have growth if there are no compound interactions, but lack growth in the experimental results). The NS scoring system was applied retroactively to the plates referred to in for FIG. 6.

The checkerboard plate data was utilized to derive a fractional inhibitory concentration index (FICI) value that scores the interaction between compounds on a three category interaction scale: FICI <0.5 indicates synergy, FICI 0.5-4 indicates no interaction, and FICI >4 indicates antagonism. The FICI equation is:

$$FICI = \frac{{MIC}_{A} in combination}{{MIC}_{A} tested alone} + \frac{{MIC}_{B} in combination}{{MIC}_{B} tested alone}$$

TABLE 1

| | Challenge Organism | | | |
|---|---|---|---|---|
| | C. albicans #ATCC 10231 | | C. glabrata # ATCC 2950 | |
| | Fluconazole | Benzethonium-Cl | Fluconazole | Benzethonium-Cl |
| Drug Composition | MIC (μg/ml) | MIC (μg/ml) | MIC (μg/ml) | MIC (μg/ml) |
| Fluconazole only | 256 | | 16 | |
| Benzethonium-Cl | | 4 | | 8 |
| only | | | | |
| Fluconazole + | 8 | 1 | 8 | 4 |
| Benzethonium-Cl | | | | |

| | | |
|---|---|---|
| FICI score | 0.28 | 1 |
| FICI category | Synergy | No interaction |

Table 1 shows the FICI calculation corresponding to the results shown in FIG. 6. Here, the NS scoring system was applied retroactively to photographs of the test plates (NS≦2 is efficacious).

TABLE 2

| | Challenge Organism | | | |
|---|---|---|---|---|
| | C. albicans #10231 | | C. glabrata #2950 | |
| | Fluconazole | Benzethonium-Cl | Fluconazole | Benzethonium-Cl |
| Drug Composition | MIC (μg/ml) | MIC (μg/ml) | MIC (μg/ml) | MIC (μg/ml) |
| Fluconazole only | 128 | | 16 | |
| Benzethonium-Cl | | 4 | | 16 |
| only | | | | |
| Fluconazole + | 8 | 1 | 2 | 8 |
| Benzethonium-Cl | | | | |

| | | |
|---|---|---|
| FICI score | 0.31 | 0.63 |
| FICI category | Synergy | No interaction |

Table 2 shows the FICI calculation corresponding to the results shown in FIG. 7.

TABLE 3

| | Challenge Organism | | | |
|---|---|---|---|---|
| | C. albicans #10231 | | C. glabrata #2950 | |
| | Fluconazole | Benzalkonium-Cl | Fluconazole | Benzalkonium-Cl |
| Drug Composition | MIC (μg/ml) | MIC (μg/ml) | MIC (μg/ml) | MIC (μg/ml) |
| Fluconazole only | 64 | | 16 | |
| Benzalkonium-Cl | | 3.8 | | 1.9 |
| only | | | | |
| Fluconazole + | 2 | 0.95 | 8 | 0.95 |
| Benzalkonium-Cl | | | | |

| | | |
|---|---|---|
| FICI score | 0.28 | 1 |
| FICI category | Synergy | No interaction |

Table 3 shows the FICI calculation corresponding to the results shown in FIG. 8.

TABLE 4

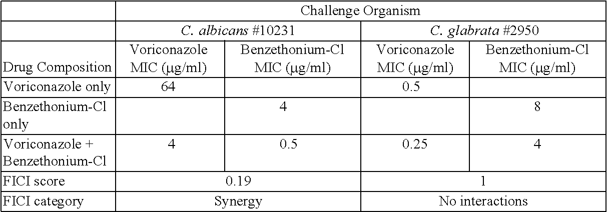

Table 4 shows the FICI calculation corresponding to the results shown in FIG. 9.

This efficacy gain is synergistic as assessed by the FICI method for the azole-quat combination against C. albicans #10231. Results for C. glabrata #2950 suggest minor gains in efficacy when combining these compounds, but results in a no interaction score using the FICI method.

Although the invention has been described and illustrated with reference to specific illustrative embodiments thereof, it is not intended that the invention be limited to those illustrative embodiments. Those skilled in the art will recognize that variations and modifications can be made without departing from the true scope and spirit of the invention as defined by the claims that follow. It is therefore intended to include within the invention all such variations and modifications as fall within the scope of the appended claims and equivalents thereof.

What is claimed is:

1. A composition comprising a quaternary ammonium salt and an azole, wherein the triazole and the quaternary ammonium salt are present in proportions providing a synergistic effect against a fungal organism.

2. The composition of claim 1, wherein the azole is a triazole.

3. The composition of claim 1, further comprising a pharmaceutically acceptable carrier.

4. The composition of claim 1, wherein the azole is selected from the group consisting of fluconazole and voriconazole.

5. The composition of claim 1, wherein the quaternary ammonium salt is selected from the group consisting of benzethonium chloride and benzalkonium chloride.

6. The composition of claim 1, wherein the azole is selected from the group consisting of fluconazole and voriconazole and wherein the quaternary ammonium salt is selected from the group consisting of benzethonium chloride to benzalkonium chloride.

7. The composition of claim 1, wherein the quaternary ammonium salt and the azole are present at a weight ratio in the range between 300:1 w/w and 1:300 w/w.

8. A method of treating or preventing an infection in a patient comprising administrating a therapeutically effective amount of a composition comprising a quaternary ammonium salt and a azole to the subject, wherein the azole and the quaternary ammonium salt are present in proportions providing a synergistic effect against a fungal infection.

9. The method of claim 8, wherein the fungal infection is an infection selected from a group consisting of a urinary tract infection, a surgical site infection, a respiratory tract infection and bloodstream infection.

10. The method of claim 9, wherein the fungal infection is caused by a species of the genus Candida.

11. The method of claim 8, wherein the administrating is systemic administrating.

12. The method of claim 8, wherein the composition is administrated as an elutable component of an implantable medical device.

13. The method of claim 12, wherein the implantable medical device is a catheter.

14. An implantable medical device comprising
a body structure comprising a polymeric material and
a composition comprising a quaternary ammonium salt and an azole,
wherein the azole and the quaternary ammonium salt are present in proportions providing a synergistic effect against a fungal organism, an wherein the composition is contained within the polymeric material in a manner that allows for controlled elution of the composition upon implantation.

15. The implantable medical device of claim 14, wherein the body structure comprises pores.

16. The implantable medical device of claim 14, wherein the implantable medical device comprises a catheter.

17. The implantable medical device of claim 14, wherein the azole is a triazole.

18. The implantable medical device of claim 17, wherein the quaternary ammonium salt is selected from the group consisting of benzethonium chloride and benzalkonium chloride.

19. The implantable medical device of claim 14, wherein the azole is selected from the group consisting of fluconazole and voriconazole and wherein the quaternary ammonium salt is selected from the group consisting of benzethonium chloride and benzalkonium chloride.

20. The implantable medical device of claim 14, wherein the polymeric material includes the quaternary ammonium salt and the azole at a ratio of between 300:1 w/w and 1:300 w/w.
